(19) United States
(12) Patent Application Publication
(10) Pub. No.: US 2007/0123540 A1
(43) Pub. Date: May 31, 2007

(54) Sexual desire enhancing medicaments comprising benzimidazolone derivatives

(76) Inventor: Angelo Ceci, Mittelbiberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/550,869

(22) Filed: Oct. 19, 2006

(30) Foreign Application Priority Data
Oct. 29, 2005 (EP) 05023718

(51) Int. Cl. A61K 31/496

(52) U.S. Cl. 514/254.06

(57) ABSTRACT

The invention relates to the use of benzimidazolone derivatives of formula (I) for the preparation of a medicament for the treatment of sexual desire disorders.

The invention relates to the use of benzimidazolone derivatives of formula (I) and their acid addition salts for the preparation of a medicament for the treatment of sexual desire disorders.

### DESCRIPTION OF THE INVENTION

The compounds of formula (I) and their acid addition salts are disclosed in WO 01/21593 A1and have the following chemical structure: 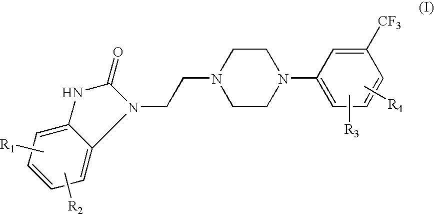
wherein R₁, R₂, R₃, and R₄ denote hydrogen or hydroxy with the proviso that R₁, R₂, R₃, and R₄ cannot simultaneously represent hydrogen.

Preferred compounds according to the present invention are those of general formula (I) wherein two or three of the four radicals R₁, R₂, R₃, and R₄ denote hydrogen. Also preferred are those compounds of general formula (I) wherein one of the radicals R₁, R₂, R₃, and R₄ denotes hydroxy, whilst the other radicals represent hydrogen.

Above mentioned compounds show affinity for the 5-HT1A and 5-HT2-receptor. They may be of value in the treatment of those diseases where an altered functioning of neurosignal transmission is present. Examples of these CNS disorders include depression, schizophrenia, Parkinson, anxiety, sleep disturbances, sexual and mental disorders and age associated memory impairment (WO 01/21593 A1).

The generic term “Sexual disorders” includes Sexual Desire Disorders, Sexual Arousal Disorders, Orgasmic Disorders, Sexual Pain Disorders, Sexual Dysfunction due to a General Medical Condition, Substance-induced Sexual Dysfunction, and Sexual Dysfunction not otherwise specified (Diagnostic and Statistical Manual of Mental Disorders, 4th edition, Text Revision. Washington DC, American Psychiatric Association, 2000).

The present invention relates to the use of the compounds of formula (I), optionally in form of the pharmacologically acceptable acid addition salts thereof for the preparation of a medicament for the treatment of sexual desire disorders which are a subgroup of sexual disorders.

In a preferred embodiment, the present invention relates to the use of the compounds of formula (I) selected from the group consisting of 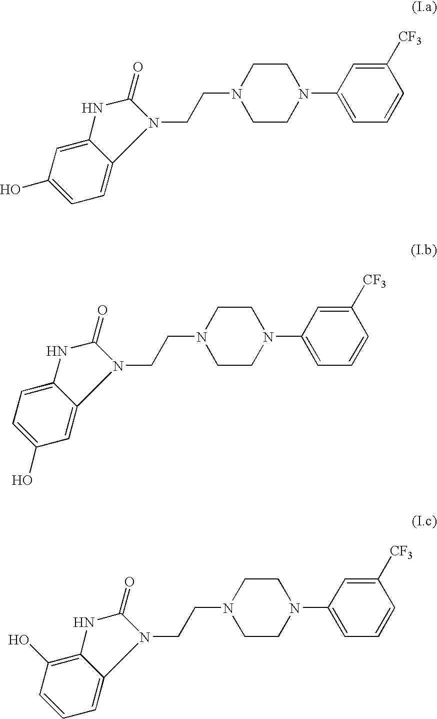 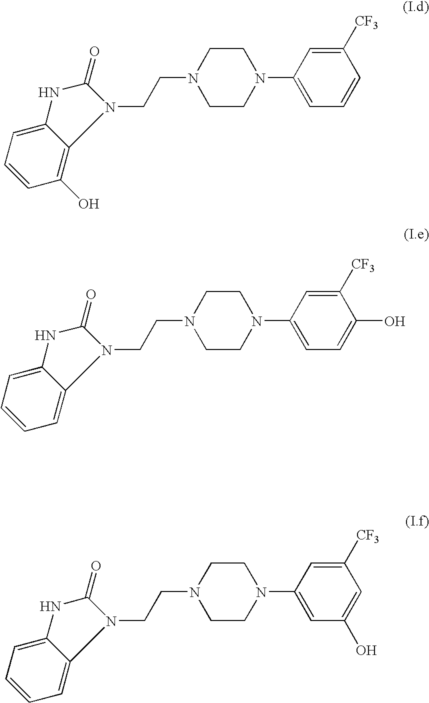 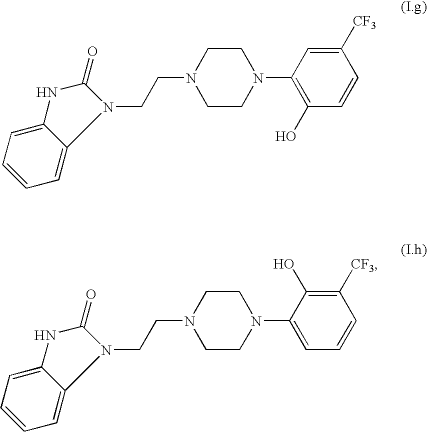
optionally in form of the pharmacologically acceptable acid addition salts thereof for the preparation of a medicament for the treatment of sexual desire disorders.

In another preferred embodiment the invention relates to the use of the compounds of formula (I), optionally in form of the pharmacologically acceptable acid addition salts thereof for the preparation of a medicament for the treatment of disorders selected from the group consisting of hypoactive sexual desire disorder, sexual aversion disorder, loss of sexual desire, lack of sexual desire, decreased sexual desire, inhibited sexual desire, loss of libido, libido disturbance, and frigidity.

In another preferred embodiment the invention relates to the use of the compounds of formula (I) selected from the group consisting of the compounds (I.a), (I.b), (I.c), (I.d), (I.e), (I.f), (I.g) and (I.h), optionally in form of the pharmacologically acceptable acid addition salts thereof for the preparation of a medicament for the treatment of disorders selected from the group consisting of hypoactive sexual desire disorder, sexual aversion disorder, loss of sexual desire, lack of sexual desire, decreased sexual desire, inhibited sexual desire, loss of libido, libido disturbance, and frigidity.

Further preferred according to the invention is the use of the compounds of formula (I), optionally in form of the pharmacologically acceptable acid addition salts thereof for the preparation of a medicament for the treatment of disorders selected from the group consisting of hypoactive sexual desire disorder, sexual aversion disorder, loss of sexual desire, lack of sexual desire, decreased sexual desire, inhibited sexual desire.

Further preferred according to the invention is the use of the compounds of formula (I), selected from the group consisting of the compounds (I.a), (I.b), (I.c), (I.d), (I.e), (I.f), (I.g) and (I.h), optionally in form of the pharmacologically acceptable acid addition salts thereof for the preparation of a medicament for the treatment of disorders selected from the group consisting of hypoactive sexual desire disorder, sexual aversion disorder, loss of sexual desire, lack of sexual desire, decreased sexual desire, inhibited sexual desire.

In another preferred embodiment the invention relates to the use of the compounds of formula (I), optionally in form of the pharmacologically acceptable acid addition salts thereof for the preparation of a medicament for the treatment of disorders selected from the group of hypoactive sexual desire disorder and loss of sexual desire.

In another preferred embodiment the invention relates to the use of the compounds of formula (I), selected from the group consisting of the compounds (I.a), (I.b), (I.c), (I.d), (I.e), (I.f), (I.g) and (I.h), optionally in form of the pharmacologically acceptable acid addition salts thereof for the preparation of a medicament for the treatment of disorders selected from the group of hypoactive sexual desire disorder and loss of sexual desire.

The observed effects of the compounds of formula (I) and the compounds (I.a), (I.b), (I.c), (I.d), (I.e), (I.f), (I.g) and (I.h), optionally in form of the pharmacologically acceptable acid addition salts thereof can be achieved in men and women. However, according to a further aspect of the invention the use of the compounds of formula (I) and the compounds (I.a), (I.b), (I.c), (I.d), (I.e), (I.f), (I.g) and (I.h), optionally in form of the pharmacologically acceptable acid addition salts for the preparation of a medicament for the treatment of female sexual desire disorders is preferred.

The beneficial effects of the compounds of formula (I) and the compounds (I.a), (I.b), (I.c), (I.d), (I.e), (I.f), (I.g) and (I.h), optionally in form of the pharmacologically acceptable acid addition salts thereof can be observed regardless of whether the disturbance existed lifelong or was acquired, is of the “generalized type” or “situational type” and independent of etiologic origin (organic—both, physically and drug induced-, psychogen (due to psychological factors), a combination of organic—both, physically and drug induced-, and psychogen (due to combined factors), or unknown). The term “lifelong” refers to such sexual disorders of the present invention, which have been present since the onset of sexual functioning. The term “acquired” refers to such sexual disorders of the present invention which developed only after a period of normal sexual functioning. The “generalized type” refers to such sexual disorders of the present invention wherein the disorder is not limited to certain types of stimulation, situations, or partners. The “situational type” applies to such sexual disorders of the present invention wherein the disorder is limited to certain types of stimulation, situations, or partners. The subtype due to “psychological factors” applies when psychological factors are judged to have the major role in the onset, severity, exacerbation, or maintenance of the sexual disorder, and general medical conditions and substance play no role in the etiology of the sexual disorder. Finally the subtype due to “combined factors” applies when 1) psychological factors are judged to have a role in the onset, severity, exacerbation, or maintenance of the sexual disorder, and 2) a general medical condition or substance use is also judged to be contributory but is not sufficient to account for a sexual disorder (Diagnostic and Statistical Manual of Mental Disorders, 4th edition, Text Revision. Washington DC, American Psychiatric Association, 2000).

The compounds of formula (I) and the compounds (I.a), (I.b), (I.c), (I.d), (I.e), (I.f), (I.g) and (I.h), can be used either as free base or in form of its pharmaceutically acceptable acid addition salts. The term “acceptable acid addition salts includes both organic and inorganic acids such as maleic, citric, tartaric, methanesulphonic, acetic, benzoic, succinic, gluconic, isethionic, glycinic, lactic, malic, mucoic, glutamic, sulphamic and ascorbic acid; inorganic acids include hydrochloric, hydrobromic, nitric, sulfuric, or phosphoric acid, and mixtures thereof.

The compounds of formula (I) and the compounds (I.a), (I.b), (I.c), (I.d), (I.e), (I.f), (I.g) and (I.h), optionally used in form of its pharmaceutically acceptable acid addition salts, may be incorporated into the conventional pharmaceutical preparation in solid, liquid or spray form. The composition may, for example, be presented in a form suitable for oral, rectal, parenteral administration or for nasal inhalation: preferred forms includes for example, capsules, tablets, coated tablets, ampoules, suppositories and nasal spray.

The active ingredient may be incorporated in excipients or carriers conventionally used in pharmaceutical compositions such as, for example, talc, arabic gum, lactose, gelatine, magnesium stearate, corn starch, acqueous or non acqueous vehicles, polyvynil pyrrolidone, semisynthetic glycerides of fatty acids, benzalconium chloride, sodium phosphate, EDTA, polysorbate 80. The compositions are advantageously formulated in dosage units, each dosage unit being adapted to supply a single dose of the active ingredient. The dosis range applicable per day is between 0.1 to 400, preferably between 1.0 to 300, more preferably between 2 to 200 mg. Each dosage unit may conveniently contain from 0,01 mg to 100 mg, preferably from 0,1 to 50 mg.

Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g. of. a flavouring such as vanilline or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection are prepared in the usual way, e.g. of. with the addition of preservatives such as p-hydroxybenzoates, or stabilisers such as alkali metal salts of ethylenediamine tetraacetic acid, and transferred into injection vials or ampoules.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

The Examples which follow illustrate the present invention without restricting its scope:

### EXAMPLES OF PHARMACEUTICAL FORMULATIONS

| A) Tablets | per tablet |
|---|---|
| compound (I. a) | 100 mg |
| lactose | 240 mg |
| corn starch | 340 mg |
| polyvinylpyrrolidone | 45 mg |
| magnesium stearate | 15 mg |
| | 740 mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

| B) Tablets | per tablet |
|---|---|
| compound (I. b) | 80 mg |
| corn starch | 190 mg |
| lactose | 55 mg |
| microcrystalline cellulose | 35 mg |
| polyvinylpyrrolidone | 15 mg |
| sodium-carboxymethyl starch | 23 mg |
| magnesium stearate | 2 mg |
| | 400 mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodium-carboxymethyl starch and the magnesium stearate are added and mixed in and the mixture is compressed to form tablets of a suitable size.

| C) Coated tablets | per coated tablet |
|---|---|
| compound (I. c) | 5 mg |
| corn starch | 41.5 mg |
| lactose | 30 mg |
| polyvinylpyrrolidone | 3 mg |
| magnesium stearate | 0.5 mg |
| | 80 mg |

The active substance, corn starch, lactose and polyvinylpyrrolidone are thoroughly mixed and moistened with water. The moist mass is pushed through a screen with a 1 mm mesh size, dried at about 45° C. and the granules are then passed through the same screen. After the magnesium stearate has been mixed in, convex tablet cores with a diameter of 6 mm are compressed in a tablet-making machine. The tablet cores thus produced are coated in known manner with a covering consisting essentially of sugar and talc. The finished coated tablets are polished with wax.

| D) Capsules | per capsule |
|---|---|
| compound (I. d) | 150 mg |
| Corn starch | 268.5 mg |
| Magnesium stearate | 1.5 mg |
| | 420 mg |

The substance and corn starch are mixed and moistened with water. The moist mass is screened and dried. The dry granules are screened and mixed with magnesium stearate. The finished mixture is packed into size 1 hard gelatine capsules.

| E) Ampoule solution | |
|---|---|
| compound (I. e) | 50 mg |
| sodium chloride | 50 mg |
| water for inj. | 5 ml |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion.

| F) Suppositories | |
|---|---|
| compound (I. f) | 50 mg |
| solid fat | 1650 mg |
| | 1700 mg |

The hard fat is melted. At 40° C. the ground active substance is homogeneously dispersed. It is cooled to 38° C. and poured into slightly chilled suppository moulds.

1. A method of treating a sexual desire disorder comprising the administration of an effective amount of a compound of formula (I) to an individual 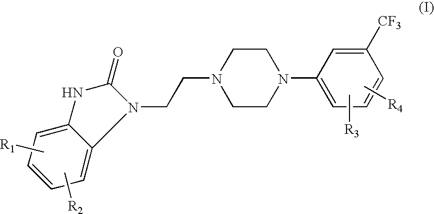
wherein R₁, R₂, R₃, and R₄ denote hydrogen or hydroxy with the proviso that R₁, R₂, R₃, and R₄ cannot simultaneously represent hydrogen, or a pharmacologically acceptable acid addition salt thereof.

2. The method according to claim 1, characterized in that the sexual desire disorder is selected from the group consisting of hypoactive sexual desire disorder, sexual aversion disorder loss of sexual desire, lack of sexual desire, decreased sexual desire, inhibited sexual desire, loss of libido, libido disturbance, and frigidity.

3. The method according to claim 2, characterized in that the sexual desire disorder is selected from the group consisting of hypoactive sexual desire disorder, sexual aversion disorder, loss of sexual desire, lack of sexual desire, decreased sexual desire, inhibited sexual desire.

4. The method according to claim 1, wherein the sexual desire disorder is a female sexual desire disorder.

5. The method according to claim 1, wherein the compound of formula (I) is in the form of a pharmaceutically acceptable acid addition salt, wherein the acid addition salt is selected from the salts formed by the acids selected from the group consisting of maleic, citric, tartaric, methanesulphonic, acetic, benzoic, succinic, gluconic, isethionic, glycinic, lactic, malic, mucoic, glutamic, sulphamic, ascorbic, hydrochloric, hydrobromic, nitric, sulfuric, phosphoric acid, and mixtures thereof.

6. The method according to claim 1, wherein the compound of formula (I) has a dosage range between 0.1 to 400 mg per day.

7. The method according to claim 1, wherein the compound of formula (I) is compound (I.a) 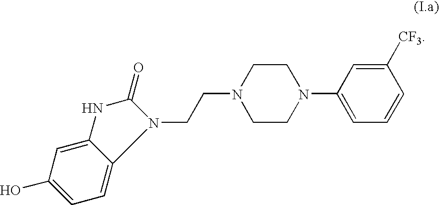
or a pharmacologically acceptable acid addition salt thereof.

8. The method according to claim 1, wherein the compound of formula (I) is compound (I.b) 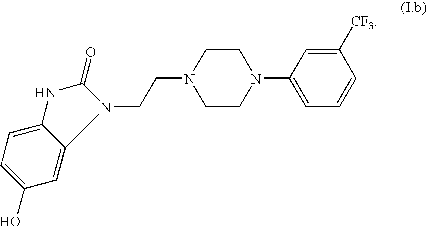
or a pharmacologically acceptable acid addition salt thereof.

9. The method according to claim 1, wherein the compound of formula (I) is compound (I.c) 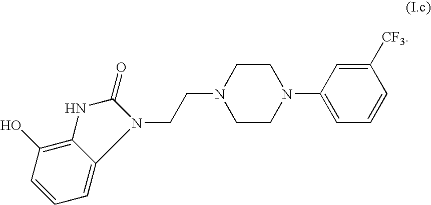
or a pharmacologically acceptable acid addition salt thereof.

10. The method according to claim 1, wherein the compound of formula (I) is compound (I.d) 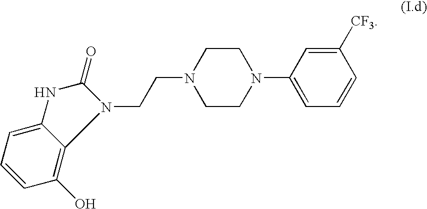
or a pharmacologically acceptable acid addition salt thereof.

11. The method according to claim 1, wherein the compound of formula (I) is 1 compound (I.e) 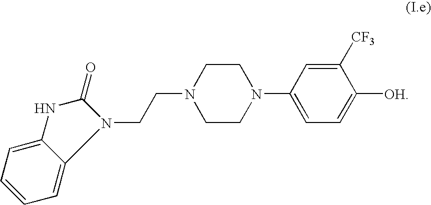
or a pharmacologically acceptable acid addition salt thereof.

12. The method according to claim 1, wherein the compound of formula (I) is compound (I.f) 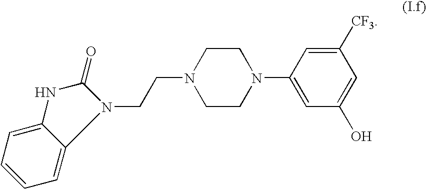
or a pharmacologically acceptable acid addition salt thereof.

13. The method according to claim 1, wherein the compound of formula (I) is compound (I.g) 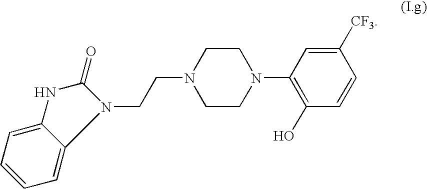
or a pharmacologically acceptable acid addition salt thereof.

14. The method according to claim 1, wherein the compound of formula (I) is compound (I.h) 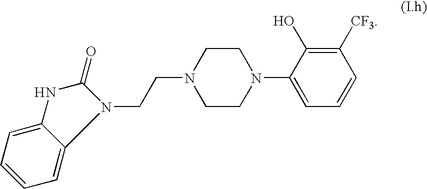
or a pharmacologically acceptable acid addition salt thereof.
